(19) United States
(12) Patent Application Publication
(10) Pub. No.: US 2013/0237597 A1
(43) Pub. Date: Sep. 12, 2013

(54) EXTENDED-RELEASE SOLID ORAL DOSAGE FORM FOR HOMEOPATHIC COMPOSITIONS

(71) Applicant: Albert Duoibes, East Grand Rapids, MI (US)

(72) Inventor: Albert Duoibes, East Grand Rapids, MI (US)

(21) Appl. No.: 13/789,311

(22) Filed: Mar. 7, 2013

Related U.S. Application Data
(60) Provisional application No. 61/607,797, filed on Mar. 7, 2012.

(51) Int. Cl. A61K 31/375

(52) U.S. Cl. CPC A61K 31/375
USPC 514/474

(57) ABSTRACT

Compositions including homeopathic active ingredients prepared in timed and/or extended-release dosage form are provided. In an embodiment, a time-release composition comprises an active ingredient component including at least one homeopathic component, and an excipient or carrier component including one or more hydrophilic polymers. A method for treating symptoms due to colds and/or flu is provided comprising administering an extended-release composition.

This application claims the benefit of earlier filed U.S. Provisional Application No. 61/607,797, filed on Mar. 7, 2012, which is hereby incorporated by reference herein.

### FIELD OF INVENTION

Compositions including homeopathic active ingredients prepared in timed and/or extended-release dosage form are provided.

### BACKGROUND

Homeopathic medicine has a documented 200 year history of being a safe and effective method of treating a variety of ailments. Homeopathic medicines are generally administered to an individual to treat the cause of the ailment and/or to ameliorate one or more symptoms associated with the ailment. Homeopathic medicine works on the Law of Infinitesimals which deals more on the sub-atomic level and the laws of physics.

As a theory, the Law of Infinitesimals works on the principle or philosophy of like curing like. In other words, substances, which if taken or ingested in a pure or full form cause certain undesirable symptoms or effects, when highly diluted or potentized will cause the opposite effects or symptoms. To simplify, ingesting less of a substance becomes more powerful than ingesting more of the same substance. Strange as it may seem, the Law of Infinitesimals and its association with treating and/or ameliorating certain ailments deals more with the imprint a substance makes upon an individual's system and the frequency of exposure to substances found primarily in nature than the actual substance itself. This is why ingestion of homeopathic medicines generally does not produce serious adverse side effects.

Homeopathic medicine actually stimulates the body's own internal mechanism to heal itself even when dealing with symptoms, as opposed to allopathic therapies (traditionally western medicine) which often succeed only in masking or covering up symptoms. Allopathic drugs may also cause serious, sometimes life threatening, side effects rather than safely and effectively treating the root cause of a disease. The body's innate ability to heal itself is a powerful force and while short of miraculous, is truly remarkable.

Typically, homeopathic medicines or formulations are orally administered in a liquid form by directly spraying or applying the composition to the mucosal tissues of the mouth or by ingestion of an aqueous solution. Homeopathic medicines, often in the pure or tincture form, may also be topically administered to the skin via a cream or gel. In some instances, homeopathic medicines may be orally administered in a solid dosage form such as a powder or sugar base medium which is made or pressed into tablets.

However, as with all medicines, in order to achieve the greatest benefit from homeopathic medicines, consistency in taking the medication regimen is critical to optimize the desired results. In most settings, practitioners rely on the diligence and competence of the patient to be cognizant of and follow recommended dosing and frequency guidelines with precision, which is hard to achieve considering that most patients are sick in the first place. Lack of diligence and/or inconsistency in following the recommended dosing regimen by missing or skipping doses or varying from the optimal timing between doses can all negatively impact the effectiveness of a medicine. Further, certain medicines need to be administered multiple times over the course of a 24 hour period in order to maintain desired levels of the active agents within the blood stream or body which also may reduce an individual's likelihood of complying with the recommended dosing regimen. While homeopathic medicine is somewhat forgiving in this regard, optimization of a remedy's effectiveness works exponentially in homeopathy. Such optimization is generally not merely routine, but may be desired empirically or anecdotally.

Accordingly, in view of the above, there is a need and a desire to provide a homeopathic medicine which can be more reliably and consistently administered to a patient or individual in need of the medicine. There is a further need and a desire for a homeopathic medicine in a dosage form which more consistently and/or reliably delivers a desired dose of active ingredients to an individual's system. There is a still further need to provide a homeopathic medicine in a dosage form which releases a regular dose of the active ingredients over an extended period of time.

### SUMMARY

A general object of the invention is to provide an extended-release dosage form including one or more homeopathic ingredients as the active agent. A further object is to provide homeopathic remedy in an extended-release dosage form.

A more specific object of the invention is to overcome one or more of the problems described above, namely consistency in administration of a dosage form, maintenance of desired blood levels of the active agents in the body, and reliable and consistent delivery of desired dosage(s) in vivo.

In one embodiment, a homeopathic remedy having an extended-release dosage form includes one or more homeopathic active ingredients and one or more release controlling polymers.

In accordance with another embodiment, a homeopathic remedy includes one or more homeopathic active ingredients dispersed in a hydrophilic excipient matrix which includes a powdered release-controlling polymer and a granular release-controlling polymer. The homeopathic remedy can be a solid oral dosage form such as a tablet. The hydrophilic excipient matrix can further include one or more additional excipients, processing aids, lubricants, fillers, and the like.

In accordance with a further embodiment, a homeopathic composition in a solid oral dosage form for use in the alleviation of one or more symptoms associated with colds and/or flu's comprises: at least one homeopathic active ingredient selected from the group consisting of Althaea officinalis, antimonium crudum, bismuthum, Bryonia alba, Calendula officinalis, Coccus cacti, condurango, dulcamara, Echinacea angustifolia Rudbeckia, Gratiola officinalis, iodium, Phytolacca decandra, Sarcolacticum acidum, Wyethia helenioides, zinc metallicum, zinc oxydatum, and combinations thereof; and a hydrophilic excipient matrix.

In accordance with yet another embodiment, a homeopathic composition in a solid oral dosage form for use in the alleviation of one or more symptoms associated attention deficit disorder (ADD) and/or attention deficit hyperactivity disorder (ADHD) comprises: at least one homeopathic active ingredient selected from the group consisting of Aconitum ferox, adrenalinum, Aesculus hippocastanum, Apis mellifica, Argentum nitricum, Avena sativa, Baptista tinctoria, Cochlearia amoracia, phosphorus, Scleranthus annuus, Scutellaria lateriflora, Sumbal, Viola odorata, and combinations thereof; and a hydrophilic excipient matrix.

### BRIEF DESCRIPTION OF THE DRAWINGS

FIG. 1 depicts time-released levels of an exemplary active ingredient (ascorbic acid) over 8 hours as measured by HPLC in a time-release tablet preparation according to one embodiment of the present invention.

### DETAILED DESCRIPTION

One or more of the above objects can be achieved, at least in part, by combining one or more homeopathic ingredients with materials which control the rate at which the active ingredients are released once ingested.

Many drugs are not inherently long lasting and require multiple daily dosing to achieve the desired therapeutic results. When conventional, immediate-release dosage forms are taken on schedule and more than once daily, there are sequential therapeutic blood peaks and valleys (troughs) associated with the taking of each dose. However, when doses are not administered on schedule, the resulting peaks and valleys reflect less than optimum drug therapy. If doses are missed, periods of subtherapeutic drug blood levels or those below the minimum effective concentration may result with no patient benefit.

Extended-release tablets and capsules are commonly taken only once or twice daily compared with counterpart conventional forms that may need to be taken three to four times daily to achieve the same therapeutic effect. Typically, extended release products provide an immediate release of drug which promptly produces the desired therapeutic effect, which is then followed by the gradual and continual release of additional amounts of drug to maintain this effect over a predetermined period of time. The sustained plasma drug levels provided by extended-release drug products often-times eliminates the need for night dosing, which provides benefits not only to the patient but to the caregiver as well.

As used herein, the term “extended release dosage forms” refers to products that are designed to release their active ingredient(s) in a controlled manner, for example, at a predetermined rate, duration and location to achieve and maintain optimum therapeutic blood levels of such ingredient(s). Extended release dosage forms may also be referred to as sustained-release, sustained-action, prolonged-action, controlled-release, time-released, and/or long-acting dosage forms.

The FDA defines an extended-release dosage form as one that allows a reduction in dosing frequency to that presented by a conventional dosage form, e.g., a solution or an immediate-release dosage form.

For a successful extended-release product, the drug must be released from the dosage form at a predetermined rate, dissolve in the gastrointestinal fluids, maintain sufficient gastrointestinal residence time, and be absorbed at a rate that will replace the amount of drug being metabolized and excreted. Thus, parameters relating to absorption, distribution, metabolism, and excretion (collectively, “ADME”) may be optimized.

In general, drugs best suited for incorporation into an extended-release product have the following characteristics: (1) they exhibit neither very slow nor very fast rates of absorption and excretion; (2) they are uniformly absorbed from the gastrointestinal tract; (3) they are administered in relatively small doses; (4) they possess a good margin of safety; and (5) they are used in the treatment of chronic rather than acute conditions.

For orally administered dosage forms, extended drug action is achieved by affecting the rate at which the drug is released from the dosage form and/or by slowing transit time of the dosage form through the gastrointestinal tract. The rate of active ingredient release from a solid dosage form, i.e., a compressed tablet, may be modified by controlling access of biological fluids to the active ingredient through the use of a barrier coating (e.g., enteric) and/or matrices which slowly erode.

Enteric coatings are often used to protect the active ingredient(s) contained within or on a core or pellet and provide a modified-release of the active ingredient as the coating dissolved or disintegrates. However, enteric coating breakdown can be uncertain and timing of when and how much of an active ingredient was released can be a difficult variable to control. In some cases, enteric coated dosage forms can traverse the gastrointestinal system and be excreted without breaking down substantially.

Another approach to controlling or extending the release of an active agent is to embed or disperse the therapeutic agent in a slowly-eroding hydrophilic matrix system. By this process, the therapeutic agent is combined and made into multi-component granules with an excipient that slowly erodes, dissolves, or dissipates in body fluids, progressively releasing the agent for absorption. In one embodiment, when the multi-component granules are mixed with granules of the therapeutic agent prepared without an excipient, the uncombined granules provide an immediate therapeutic effect whereas the agent-excipient granules provide extended therapeutic action. The granule mix may be tableted or placed in capsules for oral delivery.

Hydrophilic cellulose polymers are commonly used as the excipient base in tableted slowly-eroding matrix systems. The effectiveness of these erodible hydrophilic matrix systems is due to a successive physic-chemical process of: hydration of the cellulosic polymers; gel formation at the polymer's surface; tablet erosion; and, the subsequent and continuous release of the active agent. Hydroxypropyl methylcellulose (HPMC), an uncrosslinked linear polymer, is commonly used to provide the hydrophilic matrix. Tablets can be prepared by thoroughly distributing HPMC in a formulation, preparing granules by wet granulation or roller compaction, and manufacturing the tablets by compression.

After ingestion, the tablet is wetted by gastric fluid and the polymer begins to hydrate. A gel layer forms around the surface of the tablet and an initial quantity of the active agent is exposed and released. As water permeates further into the tablet, the thickness of the gel layer is increased and soluble active agents diffuse through the gel layer. As the outer layer becomes fully hydrated it erodes from the tablet core. Conventional hydrophilic controlled release excipients such as HPMC, hydroxypropyl cellulose, and the like are linear polymers, not chemically crosslinked, and therefore water soluble. During the drug release process these polymers dissolve and erode.

In formulating a successful slowly-eroding hydrophilic matrix system, the polymer selected for use must form a gelatinous layer rapidly enough to protect the inner core of the tablet from disintegrating too quickly after ingestion. As the proportion of polymer is increased in the formulation so is the viscosity of the gel formed with a resultant decrease in the rate of diffusion and release of the therapeutic agent.

In accordance with one embodiment, a homeopathic formulation in the form of an extended-release solid oral dosage can include one or more homeopathic active ingredients combined with or dispersed in a slowly-eroding hydrophilic matrix. The extended-release solid oral dosage may be a compressed tablet. Alternatively, the extended-release solid oral dosage may be in the form of a capsule including a gelatin shell which contains the combined homeopathic active ingredient(s) and the slowly-eroding hydrophilic matrix.

In accordance with certain embodiments, the homeopathic formulation includes an actives portion which constitutes about 50% to about 70% by weight of the total weight of the homeopathic formulation and an excipient or carrier portion which constitutes about 30% to about 50% of the total weight of the homeopathic formulation.

Optionally, the actives portion comprises one or more homeopathic active ingredients selected to elicit complementary effects which reduce and/or alleviate the symptoms associated with a particular disease or condition. The actives portion can further include one or more of enzymes, botanical ingredients or extracts, probiotics, and combinations thereof. Each component of the actives portion of the homeopathic formulation can be present in a potency of 10×. Alternatively or additionally, each component of the actives portion of the homeopathic formulation can be present in an equal amount.

Optionally, suitable enzymes for use in the active portion of the homeopathic formulation include, but are not limited to, Amylase, Protease, Lipase, Cellulase, Invertase, Lactase, Maltase, Glucoamylase, Alpha-galactosidase, Phytase/pectinase, Xylanase, Hemicellulase, Beta-glucanase, Betaine HCl, Bison bile/ox bile, Pepsin 1:10,000, Bromelain, Papaya leaf, Pancreatin, Porcine bile extract, Papain, and combinations thereof.

Optionally, suitable botanical ingredients or extracts for use in the active portion of the homeopathic formulation include, but are not limited to, Peppermint, Aloe vera gel, Ginger, and combinations thereof.

Optionally, suitable probiotics for use in the active portion of the homeopathic formulation include, but are not limited to, Lactobacillus acidophilus, L. bulgaricus, L. casei, L. lactis, L. paracasei, L. plantarum, L. rhamnosus, L. salivarius, Bifidobacterium bifidum, B. infantis, B. longum, B. breve, Bacillus subtilus, Pediococcus acidilactici, Lactococcus lactis, and combinations thereof.

The excipient or carrier portion of the homeopathic formulation may include one or more linear, uncrosslinked hydrophilic polymers selected from the group consisting of hydroxypropyl methylcellulose (HPMC), hydroxypropyl cellulose, and combinations thereof. Suitably, the linear, uncrosslinked hydrophilic polymers can constitute about 5% to about 30% by weight of the total homeopathic formulation.

Other suitable polymers for the excipient or carrier portion of the homeopathic formulation may include one or more lightly crosslinked polymers of acrylic acid.

The excipient or carrier portion can further include one or more fillers, binders, disintegrants, lubricants, or other processing aids such as, but not limited to, pectin, microcrystalline cellulose, sodium carbonate, and/or silicon dioxide.

In accordance with one embodiment, an extended release tablet can include one or more homeopathic active ingredients combined with or dispersed in a slowly-eroding hydrophilic matrix wherein the tablet has a dosage unit weight of about 500 milligrams to about 1100 milligrams.

As described herein, the homeopathic active ingredients can be present in a potency of 4× to 400×. Alternatively or additionally, one or more of the homeopathic active ingredients in a low potency of LM-1, 2, or 3. In accordance with one aspect, each homeopathic active ingredient present in the homeopathic composition may be in medium potency of 10×. The extended release tablet can include about 50% to about 70% by weight, and suitably about 60% by weight, active ingredients based on the total dosage unit weight.

Homeopathic remedies are made from natural components—mineral, animal, and plant extracts are the base of these natural remedies which are then diluted through altering the degree of concentration to avoid creating side effects that can be disagreeable. Paradoxically, it is postulated that the more a homeopathic remedy is diluted, the more effectively a remedy will work.

There is a very distinct process for making homeopathic remedies. For example, when making a homeopathic remedy which is of plant or animal nature the extract or herb material, which generally includes a soluble substance or substances (or, alternatively, is admixed with another soluble substance), is dissolved in a mixture of alcohol (ethanol) and water (approximately ninety percent pure alcohol and ten percent distilled water, although the ratio can vary). This mixture is optionally set aside for two to four weeks. It is periodically shaken and then is press strained once it has cured. This formulation procedure yields a remedy referred to as a “mother tincture.” See the references that follow, for example, regarding preparation of mother tinctures.

As used herein the terms “homeopathic” and “homeopathic ingredient” are defined in accordance with The Federal Food, Drug and Cosmetic Act (21 U.S.C. 351), which is incorporated herein by reference, and includes those agents or drugs used in the practice of homeopathy and listed in the Homeopathic Pharmacopeia of the United States (HPUS), the entirety of which is also incorporated herein by reference.

Also incorporated herein by reference are the following homeopathic texts: “Materia Medica with Repertory,” 9^{th} Edition, by William Boericke, M.D. (Boericke & Tafel, Santa Rosa, Calif., 1927) and “A Dictionary of Practical Materia Medica,” reprinted 2006, by John Henry Clarke, M.D. (B. Jain Publishers, New Delhi, 2006). Mother tincture example (as in Clarke): Bryonia alba (white bryony) tincture of root procured before flowering, Vol. I, p. 310.

The mother tincture is diluted to produce different remedy potencies. To do this one may use one of two scales: the decimal (x or “X”) and the centesimal (c or “C”). Optionally, an alcohol/water mixture is used for dilution in various stages.

Between each of the stages of dilution, the diluted tincture is optionally succussed (shaken vigorously). Also, the decimal scale dilution factor (X) is 1:10 and in the centesimal (C) it is 1:100.

For example: To produce a 1C potency of the Bryonia alba remedy, one drop of the mother tincture is added to 99 drops of an alcohol/water mixture and succussed. To produce a 2C potency, one drop of the 1C mixture is added to 99 drops of an alcohol/water mixture and succussed. The number of a homeopathic remedy shows how many times it has been diluted and succussed, for example, Bryonia 6C has been diluted and succussed six times. Standard dilutions include 6×, 12×, and 30×.

Without being bound by theory, it is noted that statistical and probability considerations may apply in a given solute/solution combination where dilutions reach or exceed 24× (1/10²⁴ dilution), in comparison with Avogadro's number. The embodiments as described are not intended to be limited merely by theoretical calculations, particularly where a statistical probability may apply.

A further approach to controlling or extending the release of an active agent is to embed or disperse the therapeutic agent in a hydrophilic microgel-forming polymeric matrix system. By this process, the therapeutic agent is combined and made into granules with an excipient that forms a hydrogel comprising discrete microgel particles (microgels) made up of a plurality of polymer particles in body fluids, progressively releasing the agent for absorption. When the granules are mixed with granules of the therapeutic agent prepared without the excipient, the uncombined granules provide the immediate therapeutic effect whereas the agent-excipient granules provide extended therapeutic action. The granule mix may be tableted or placed in capsules for oral delivery.

Hydrophilic microgel-forming polymeric matrix systems, in contrast to slowly-eroding hydrophilic matrix systems, utilize crosslinked carbomer homopolymers that do not dissolve and erode in water. Because the crosslinked carbomer homopolymers only swell in water, when the hydrogel is fully hydrated osmotic pressure from within may break up the structure, essentially by sloughing off discrete pieces of hydrogel. Therapeutic agent release rates are affected by differences in the rates of hydration and swell of the polymer hydrogel, which are dependent upon the molecular structure of the polymers, including crosslink density, chain entanglement, and crystallinity of the polymer matrix. In the case of highly crosslinked carbomer homopolymers the release is faster as the therapeutic agent diffuses out through the water-filled interstitial spaced between the microgels. In systems using lightly crosslinked carbomer homopolymers, the microgels form a more uniform and continuous structure resulting a slower therapeutic agent release. Further, due to their crosslinked structures, hydrophilic microgel-forming polymers can form strong matrices at low concentrations.

Crosslinked carbomer homopolymers suitable for use in an extended-release homeopathic solid oral dosage form, i.e., a tablet or a capsule, include synthetic high-molecular weight polymers of acrylic acid that are crosslinked with either allyl sucrose or with allyl ethers of pentaerythritol. They contain from 56 to 68% of carboxylic acid groups, when calculated on a dry basis. Their molecular mass cannot be directly evaluated due to the presence of the crosslinker, but is estimated from 700,000 to 4 billion. Carbomers disperse (and generally do not dissolve) in water to form acidic colloidal solutions of low viscosity, however, when neutralized at above the pK value (approximately at pH>5.5), they produce highly viscous gels. Such crosslinked carbomer homopolymers can be obtained, for example, from The Lubrizol Corporation of Wickliffe, Ohio, USA under the trade names CARBOPOL® 971P NF (powder) and CARBOPOL® 71G NF (granular). The foregoing polymers are considered as release-controlling polymers.

In accordance with one embodiment, a homeopathic formulation in the form of an extended-release solid oral dosage can include one or more homeopathic active ingredients combined with or dispersed in a hydrophilic microgel-forming polymer matrix. The extended-release solid oral dosage may be a compressed tablet. Alternatively, the extended-release solid oral dosage may be in the form of a capsule including a gelatin shell which contains the combined homeopathic active ingredient(s) and the hydrophilic microgel-forming polymer matrix.

In accordance with certain embodiments, the homeopathic formulation includes an actives portion which constitutes about 50% to about 70% by weight of the total weight of the homeopathic formulation and an excipient or carrier portion which constitutes about 30% to about 50% of the total weight of the homeopathic formulation. Optionally, 10% by weight of the actives portion may be applied to rice maltodextrin, or other suitable substrate.

The actives portion comprises one or more homeopathic active ingredients selected to elicit complementary effects which may treat, prevent, reduce, and/or alleviate the symptoms associated with a particular disease or condition. The actives portion can further include one or more of enzymes, botanical ingredients or extracts, probiotics, and combinations thereof as described above. Each component of the actives portion of the homeopathic formulation can be present in a potency of 10×. Alternatively or additionally, each component of the actives portion of the homeopathic formulation can be present in an equal amount.

The excipient or carrier portion of the homeopathic formulation includes one or more crosslinked carbomer homopolymers. Suitably, the crosslinked carbomer homopolymers can constitute about 5% to about 30% by weight of the total homeopathic formulation. The excipient or carrier portion can further include one or more fillers, binders, disintegrants, lubricants, or other processing aids such as, but not limited to, pectin, microcrystalline cellulose, sodium carbonate, and/or silicon dioxide.

A granular release-controlling polymer can be used in the homeopathic formulation. In accordance with one aspect, the homeopathic formulation can include about 10% to about 30% by weight of a granular crosslinked carbomer homopolymer such as, for example, CARBOPOL® 71G NF based on the total weight of the homeopathic formulation. A powdered release-controlling polymer can be used in the homeopathic formulation. Alternatively, the homeopathic composition can include about 5% to about 30% by weight of a powdered crosslinked carbomer homopolymer such as, for example, CARBOPOL® 971P NF based on total weight of homeopathic composition.

In accordance with another aspect, the excipient or carrier portion of the homeopathic composition can include a combination of a granular crosslinked carbomer homopolymer such as, for example, CARBOPOL® 71G NF and a powdered crosslinked carbomer homopolymer such as, for example, CARBOPOL® 971P NF. For example, a homeopathic composition in accordance with embodiments herein can include about 5% by weight of a powdered crosslinked carbomer homopolymer such as, for example, CARBOPOL® 971P NF and about 20% by weight of a granular crosslinked carbomer homopolymer such as, for example, CARBOPOL® 71G NF based on the total weight of the homeopathic formulation.

In accordance with a further aspect, the excipient or carrier portion can contain both a crosslinked carbomer homopolymer such as CARBOPOL® 971P NF (powder) and/or CARBOPOL® 71G NF (granular) and an uncrosslinked linear hydrophilic polymer selected from the group consisting of hydroxypropyl methylcellulose (HPMC), hydroxypropyl cellulose, and combinations thereof.

In accordance with one embodiment, an extended release tablet can include one or more homeopathic active ingredients combined with or dispersed in a Hydrophilic microgel-forming polymeric matrix wherein the tablet has a dosage unit weight of about 500 milligrams to about 1100 milligrams. The homeopathic active ingredients can be present in a potency of 4× to 400×. Alternatively or additionally, one or more of the homeopathic active ingredients in a low potency of LM-1, 2, or 3. In accordance with one aspect, each homeopathic active ingredient present in the homeopathic composition may be in medium potency of 10×. The actives portion of the homeopathic formulation which contains the homeopathic active ingredients can constitute about 50% to about 70% by weight, and suitably about 60% by weight, of the dosage unit weight.

The foregoing description of various homeopathic formulations can be further understood by reference to following examples. While homeopathic formulations in the form of extended release solid oral dosage forms for use in the amelioration or relief of symptoms associated with colds, flu's, or other viral or microbial infections or for use in the amelioration or relief of symptoms associated with attention deficit disorder (ADD) and/or attention deficit hyperactivity disorder (ADHD) are describe below, it should be understood that other extended release solid oral dosage forms which incorporate homeopathic active ingredients, which may or may not be disclosed herein, for use in the treatment and/or amelioration of these and other conditions such as, for example, anxiety disorders, obsessive-compulsive disorders (OCD), asthma, and/or others, may be prepared in a similar manner.

In accordance with one embodiment, a homeopathic formulation in the form of an extended release solid oral dosage includes one or more homeopathic active ingredients associated with the amelioration or relief of symptoms associated with colds, flu's, or other viral or microbial infections and a hydrophilic polymer matrix including at least one polymer selected from the group consisting of uncrosslinked linear hydrophilic polymers, crosslinked carbomer homopolymers, and combinations thereof.

A number of homeopathic remedies, medicines, or active ingredients have been identified as having efficacy for the treatment and/or amelioration of symptoms associated with various types of viral or microbial infections such as the common cold.

Althaea Officinalis is often used to soothe the bronchial tubes and relieve irritation of the mucous membranes.

Antimonium Crudum may be used to relieve diarrhea, nausea and vomiting.

Bismuthum is typically used to relieve irritations and catarrhal inflammation of the alimentary canal, gastralgia, vomiting and diarrhea.

Bryonia Alba can be administered to relieve muscular aches and pains and dry hacking cough.

Calendula Officinalis is often used to relieve inflammation, heartburn, nausea, vomiting, and epigastric distention.

Coccus Cacti is believed to relieve spasmodic whooping coughs, catarrhal conditions and the accumulation of thick viscid mucus.

Condurango stimulates the digestive function and improves general health.

Dulcamara can be used to relieve nasal congestion, nausea, vomiting of mucus, chills, hoarse spasmodic whooping cough with excessive secretion of mucus and difficult breathing.

Echinacea Angustifolia Rudbeckia may be administered to ameliorate lymphatic inflammation, post-nasal catarrh, belching, heartburn, nausea and chills.

Gratiola Officinalis has been used to treat chronic catarrhal conditions, dyspepsia, cramps, colic and constipation.

Iodium is believed to relieve acute exacerbation of chronic inflammation, acute catarrh of the mucus membranes, sudden violent influenza symptoms and croupy cough.

Phytolacca Decandra is a homeopathic remedy which can be administered to relieve aching soreness, pains like shocks, restlessness, difficult breathing, dry hacking cough, and/or high fever alternating with chills.

Sarcolacticum Acidum is often used to relieve the most violent forms of epidemic influenza symptoms, nausea and uncontrollable vomiting.

Wyethia Helenioides is typically administered to ameliorate belching, hiccough, nausea, vomiting, dry hacking cough and has a marked effect on the throat rendering making it an excellent remedy in pharyngitis.

Zinc Metallicum is thought to relieve hiccough, nausea, vomiting of bitter mucus, dyspepsia, hoarseness, debilitating spasmodic cough, and bronchitis with constriction of chest.

Zinc Oxydatum may be used to relieve nausea, vomiting of bile, diarrhea and flatulence.

In accordance with another embodiment, a homeopathic formulation in the form of an extended release solid oral dosage includes one or more homeopathic active ingredients associated with the amelioration or relief of symptoms associated with attention deficit disorder (ADD) and/or attention deficit hyperactivity disorder (ADHD) and a hydrophilic polymer matrix including at least one polymer selected from the group consisting of uncrosslinked linear hydrophilic polymers, crosslinked carbomer homopolymers, and combinations thereof.

A number of homeopathic remedies, medicines, or active ingredients have been identified as having efficacy for the treatment and/or amelioration of symptoms associated with ADD and/or ADHD.

Aconitum ferox may relieve racing or overactive active mind/thoughts and difficulty comprehending written and/or mathematical materials.

Adrenalinum is believed to relieve or ameliorate a disinclination toward mental work, fidgeting, and difficulties in concentrating on task at hand.

Aesculus hippocastanum may relieve restlessness, impulsiveness, forgetfulness, difficulties reading and writing, impatience and suggestibility.

Apis mellifica is believed to provide relief from absent-mindedness, forgetfulness, lack of concentration, impaired ability to read and write, stupor, irritability, excitability, and fidgeting.

Argentum nitricum is believed to reduce the incidence of panic and/or anxiety attacks, nervous, impulsive, and hurried actions or speech, loquaciousness, and negativity.

Avena sativa may provide relief from mental exhaustion, mental exertion aggravates, difficulty thinking and comprehending, and the ability to keep mind on any one subject.

Baptista tinctoria may improve a dull and confused mind, inability to think, and mental focus.

Cochlearia amoracia is believed to provide relief from anxiety, indecision, and alternating manic and depressive moods.

Phosphorus may reduce forgetfulness, scattered thought, and feeling of mental fatigue.

Scleranthus annuus has been used to ease inner uncertainty, preoccupation, and feelings of confusion and/or being overwhelmed, capriciousness, mood swings, and sudden changes in interest.

Scutellaria lateriflora is believed to provide relief from or ameliorate mental confusion, irritability, an inability to study or fix the attention on one's work.

Sumbal has been used to reduce fidgeting, mistakes in writing and adding, and sudden mood swings.

Viola odorata is believed to improve concentration and reduce inclinations toward weeping or childish behavior.

In accordance with one embodiment, an extended release tablet can have a dosage unit weight of about 500 milligrams to about 1100 milligrams. The homeopathic active ingredients can be present in a potency of 4× to 400×. Alternatively or additionally, one or more of the homeopathic active ingredients in a low potency of LM-1, 2, or 3. In accordance with one aspect, each homeopathic active ingredient present in the extended release tablet may be in medium potency of 10×. An actives portion of the tablet which contains the homeopathic active ingredients can constitute about 50% to about 70% by weight, and suitably about 60% by weight, of the dosage unit weight.

The extended release tablet also includes an excipient or carrier portion which constitutes about 30% to about 50% by weight of the dosage unit weight and which further includes the hydrophilic polymer matrix which can constitute about 5% to about 30% by weight of the dosage unit weight. The excipient or carrier portion can further include one or more fillers, binders, disintegrants, lubricants, or other processing aids such as, but not limited to, pectin, microcrystalline cellulose, sodium carbonate, and/or silicon dioxide.

### Example

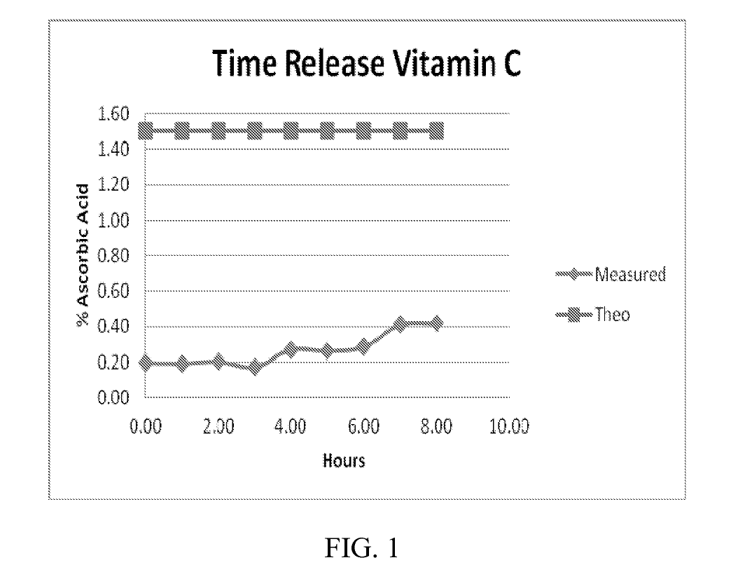

All sample measurements were made by employing HPLC standard methods. As shown in FIG. 1, a tablet prepared according to the principles of the present invention, containing a homeopathic ingredient (10×), a crosslinked carbomer homopolymer, and Vitamin C. In particular, extended release tablets having a unit dosage weight per tablet of about 500 milligrams was prepared with a homeopathic actives portion which constitutes about 68% by weight of the unit dosage weight, Vitamin C (L-ascorbic acid) which constitutes about 1.5% by weight of the unit dosage weight, and an excipient portion which constitutes about 30.5% by weight of the unit dosage weight. The actives portion of the tablet included about 10% by weight homeopathic active ingredients (10×) based on the total weight of the actives portion loaded onto a rice maltodextrin carrier. The excipient portion of the tablet included about 15% by weight crosslinked carbomer homopolymer based on the unit dosage weight. The crosslinked carbomer homopolymer included Carbopol® 971P NF and Carbopol® 71G NF in a weight:weight ratio of about 1:2. Inactive ingredients, constituting about 15.5% of the unit dosage weight, present in the excipient portion of the tablet included microcrystalline cellulose, silica dioxide, fructooligosaccharide, and magnesium stearate. The tablets prepared using standard compression tableting procedures and equipment using a compression force of about 3,500 psi without precompression, an ejection force of about 25 pounds, and a press speed of about 30-50 rpm. The resulting provide extended release tablets had an average thickness of about 0.143 inches (3.63 millimeters), an average length of about 1.5 inches, an average hardness of about 10.9 Strong Cobb Units (SCU), and a friability of less than 0.01%.

In order to determine dissolution rate, 750 mL of 0.1 N HCl were added to a 2 Liter beaker. Two (2) whole tablets were added to the beaker and an initial 0.5 mL sample was removed. The tablets were gently stirred (60 RPM) with a magnetic stirrer and Teflon coated stir bar. A 0.5 mL sample was removed each hour and placed in a brown HPLC vial and capped. Samples were stored at 4° C. prior to analysis. Measured values are shown in FIG. 1 as measured % ascorbic acid/theoretical over the course of 8 hours. It was shown that the tablet prepared in accordance with the described embodiment exhibits good time-release properties, providing significant measured levels of an active component (Vitamin C) released in solution.

As shown in the example, it is expected that a tablet administered to a human individual afflicted with an infection resulting from a cold and/or flu virus would benefit from the time-release properties of the tablet. That is, a time-release tablet composition of Example 1 would be expected to provide relief from symptoms caused by cold and/or flus.

In accordance with certain embodiments, a homeopathic formulation in the form of extended release can include one or more homeopathic active ingredients utilizing several delivery systems that can optimally deliver drugs across the skin and mucosa. Using active transdermal technology that uses an array of plastic microstructures to mechanically create reversible “micro-pores” in the stratum corneum to enhance transdermal delivery, especially for large molecules like proteins and vaccines that are typically administered by injection. polymer technology that can be delivered in liquid, semi-solid and solid formats for conventional transdermal, mucosal delivery. These are suitable for rapid and sustained drug release and can be used in the development of pharmaceutical products with acute and chronic applications.

In accordance with another embodiment, an extended-release homeopathic product in a liquid form can include one or more homeopathic active ingredients combined with or dispersed in a slowly releasing liquid matrix wherein tiny particles coated with the homeopathic component to be delivered over the time dosage span are suspended in a liquid.

In accordance with another embodiment, an extended release homeopathic product can be combined in a bi-layer tablet form that includes one or more homeopathic or non homeopathic drug actives (such as, but not limited to acetaminophen, ibuprofen, and the like). The layer containing the non homeopathic drug active component provides immediate release via “fast melt” technology, and symptom relief, while the extended release of the homeopathic components, contained in the other half of the tablet provides relief over time.

In accordance another embodiment, an extended release homeopathic product can include a coating of a tablet as in using an enteric coating to protect one or more active homeopathic ingredients contained within a pellet or tablet core.

In accordance with a further embodiment, an extended-release tablet for use in the alleviation of one or more symptoms associated with colds and/or flu's comprises:
(a) an active ingredient portion including at least one homeopathic active ingredient selected from the group consisting of Althaea Officinalis, antimonium crudum, bismuthum, Bryonia alba, Calendula officinalis, Coccus cacti, condurango, dulcamara, Echinacea angustifolia Rudbeckia, Gratiola officinalis, iodium, Phytolacca decandra, Sarcolacticum acidum, Wyethia helenioides, zinc metallicum, zinc oxydatum, and combinations thereof, at least one enzyme selected from the group consisting of Amylase, Protease, Lipase, Cellulase, Invertase, Lactase, Maltase, Glucoamylase, Alpha-galactosidase, Phytase/pectinase, Xylanase, Hemicellulase, Beta-glucanase, Betaine HCl, Bison bile/ox bile, Pepsin 1:10,000, Bromelain, Papaya leaf, Pancreatin, Porcine bile extract, Papain, and combinations thereof, at least one botanical ingredients or extracts selected from the group consisting of Peppermint, Aloe vera gel, Ginger, and combinations thereof, and at least one probiotic selected from the group consisting of Lactobacillus acidophilus, L. bulgaricus, L. casei, L. lactis, L. paracasei, L. plantarum, L. rhamnosus, L. salivarius, Bifidobacterium bifidum, B. infantis, B. longum, B. breve, Bacillus subtilus, Pediococcus acidilactici, Lactococcus lactis, and combinations thereof; and
(b) an excipient or carrier portion comprising a hydrophilic microgel-forming polymeric matrix system including a crosslinked carbomer homopolymer selected from the group consisting of granular crosslinked carbomer homopolymers, powdered crosslinked carbomer homopolymers, and combinations thereof.

Each ingredient in the active portion may be present in a potency of 10×. The excipient or carrier portion of the extended-release tablet can further comprises one or more uncrosslinked linear hydrophilic polymers selected from the group selected from hydroxypropyl methylcellulose (HPMC), hydroxypropyl cellulose, and combinations thereof. The excipient or carrier portion of the extended-release tablet can additionally include one or more inactive ingredient selected from the group consisting of pectin, microcrystalline cellulose, sodium carbonate, and/or silicon dioxide.

A method for alleviating one or more symptoms associated with associated with colds and/or flu's includes administering to an individual in need thereof a therapeutically effective dose of a homeopathic extended-release solid oral dosage form as described herein.

The method can further include administering a loading dose which includes ingesting a first dose of one to two homeopathic extended-release solid oral dosage forms at the onset of symptoms and ingesting a second dose of one to two homeopathic extended-release solid oral dosage 4 hours after the first dose. Thereafter, additional doses can be administered at 12 hour intervals, e.g., morning and evening, until symptoms are gone. The method can also include administering a dose of the homeopathic formulation to an individual in need thereof by placing one or more homeopathic extended-release solid oral dosage forms in the oral cavity, holding the dose in the mouth for approximately 30 seconds to partially hydrate the hydrophilic microgel-forming polymeric matrix on an exterior surface of the homeopathic extended-release solid oral dosage form, and swallowing the homeopathic extended-release solid oral dosage form.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

The use of the terms “a,” “an,” “the,” and similar referents in the context of describing the presently claimed invention (especially in the context of the claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Use of the term “about” is intended to describe values either above or below the stated value in a range of approx.±10%; in other embodiments the values may range in value either above or below the stated value in a range of approx.±5%; in other embodiments the values may range in value either above or below the stated value in a range of approx.±2%; in other embodiments the values may range in value either above or below the stated value in a range of approx.±1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

I claim:

1. An extended-release homeopathic composition comprising an active ingredient component and an excipient component.

2. The extended-release homeopathic composition according to claim 1, wherein the active ingredient includes at least one homeopathic component.

3. The extended-release homeopathic composition according to claim 2, wherein the excipient component includes one or more hydrophilic polymers selected from the group consisting of a granular crosslinked carbomer homopolymer, a powdered crosslinked carbomer homopolymer, and combinations thereof.

4. The extended-release homeopathic composition according to claim 3, wherein the one or more hydrophilic polymers comprise a microgel-forming polymeric matrix.

5. The extended-release homeopathic composition according to claim 3, wherein the granular crosslinked carbomer homopolymer is present in an amount of about 10% by weight to about 30% by weight based on the total weight of the composition, and wherein the powdered crosslinked carbomer homopolymer is present in an amount of about 5% by weight to about 30% by weight based on the total weight of the composition.

6. The extended-release homeopathic composition according to claim 3, wherein the homeopathic component is present in an amount of about 50% by weight to about 70% by weight based on the total weight of the composition.

7. The extended-release homeopathic composition according to claim 6, wherein the homeopathic component is selected from the group consisting of Althaea Officinalis, antimonium crudum, bismuthum, Bryonia alba, Calendula officinalis, Coccus cacti, condurango, dulcamara, Echinacea angustifolia Rudbeckia, Gratiola officinalis, iodium, Phytolacca decandra, Sarcolacticum acidum, Wyethia helenioides, zinc metallicum, zinc oxydatum, and combinations thereof.

8. The extended-release homeopathic composition according to claim 7, wherein the homeopathic component is present in a dilution from about 4× to about 400×.

9. The extended-release homeopathic composition according to claim 8, wherein the homeopathic component is present in a dilution of about 10×.

10. The extended-release homeopathic composition according to claim 1 which is in a solid oral dosage form.

11. The extended-release homeopathic composition according to claim 10, wherein said solid oral dosage form is a tablet.

12. The extended-release homeopathic composition according to claim 11, wherein said tablet has a dosage unit weight of from about 500 mg to about 1100 mg.

13. A method of treating or preventing the symptoms of colds and/or flus in an individual comprising the steps of:
(a) providing an extended-release homeopathic composition comprising at least one homeopathic component selected from the group consisting of Althaea officinalis, antimonium crudum, bismuthum, Bryonia alba, Calendula officinalis, Coccus cacti, condurango, dulcamara, Echinacea angustifolia Rudbeckia, Gratiola officinalis, iodium, Phytolacca decandra, Sarcolacticum acidum, Wyethia helenioides, zinc metallicum, zinc oxydatum, and combinations thereof; and an excipient component including one or more hydrophilic polymers selected from the group consisting of a granular crosslinked carbomer homopolymer, a powdered crosslinked carbomer homopolymer, and combinations thereof; and
(b) administering the extended-release homeopathic composition orally to the individual in need of such treatment;
wherein the symptoms are decreased.

14. The method of claim 13, wherein the hydrophilic polymers comprise a microgel-forming polymeric matrix.

15. The method of claim 14, wherein the homeopathic component is present in an amount of about 50% by weight to about 70% by weight based on the total weight of the composition.

16. The method of claim 14, wherein the granular crosslinked carbomer homopolymer is present in an amount of about 10% by weight to about 30% by weight based on the total weight of the composition, and wherein the powdered crosslinked carbomer homopolymer is present in an amount of about 5% by weight to about 30% by weight based on the total weight of the composition.

17. The method of claim 14, wherein the homeopathic component is present in a dilution from about 4× to about 400×.

18. The method of claim 14, wherein the cold and/or flu symptoms are decreased after about 24 hours to about 48 hours.

19. The method of claim 18, wherein the cold and/or flu symptoms include fever.

20. The method of claim 18, wherein the cold and/or flu symptoms include nausea.
